(19) United States
(12) Patent Application Publication
(10) Pub. No.: US 2006/0115524 A1
(43) Pub. Date: Jun. 1, 2006

(54) Method for preparation of an agglomerate using melt agglomeration

(75) Inventor: Helle Eliasen, Koge (DK)

Correspondence Address: DARBY & DARBY P.C., P. O. BOX 5257, NEW YORK, NY, 10150-5257, US

(73) Assignee: H. Lundbeck A/S, Valby-Copenhagen (DK)

(21) Appl. No.: 10/543,789

(22) PCT Filed: Feb. 18, 2004
(86) PCT No.: PCT/DK04/00111

§ 371 Date: Jul. 28, 2005

(30) Foreign Application Priority Data
Feb. 19, 2003 (DK) PA 2003 00252

(51) Int. Cl. A61K 9/48; A61K 9/20; B27N 3/00

(52) U.S. Cl. 424/451; 424/464; 264/109

(57) ABSTRACT

Melt agglomeration for the preparation of an agglomerated material is performed us-ing a vehicle that can be melted at a suitable temperature, an active compound and silicon dioxide as filler. Using silicon dioxide as a filler in melt agglomeration has the benefit that a high ratio of melt to filler may be used. Agglomerates prepared by the process proved to be readily compressible into tablets using common equipment. Melt agglomeration for the preparation of an agglomerated material is performed us-ing a vehicle that can be melted at a suitable temperature, an active compound and silicon dioxide as filler. Using silicon dioxide as a filler in melt agglomeration has the benefit that a high ratio of melt to filler may be used. Agglomerates prepared by the process proved to be readily compressible into tablets using common equipment.

The invention relates to agglomerated compositions comprising particulate silicon dioxide as a filler, a meltable vehicle and a pharmaceutical active compound. The agglomerated compositions according to the invention are useful for the preparation of solid pharmaceutical medicaments for oral administration.

### BACKGROUND FOR THE INVENTION

In the pharmaceutical area it is common to prepare pharmaceutical compositions comprising one or more active compounds and various excipients. One reason for preparing such pharmaceutical compositions is to manipulate the availability of the active compound after ingestion of the pharmaceutical composition.

For the preparation of pharmaceutical compositions for oral administration the active compounds are often incorporated into an agglomerated preparation in order to provide the active compounds in a form that may be pressed into tablets or filled into capsules.

Beside providing the active compound in a form that may be pressed into tablets, agglomerates may also be designed to secure a desired availability of the active compound after ingestion of a pharmaceutical composition containing said granule.

One commonly used technique for granulation is wet granulation, where a mixture of powders including the active compound is mixed with a liquid, usually an aqueous liquid, under mechanical influence for the preparation of granules. Usually the granules prepared by wet granulation are dried before use.

Melt agglomeration is a technique for agglomeration of one or more active compounds, a filler and optional excipients with a pharmaceutical acceptable vehicle having a melting point above ambient temperatures comprising
bringing the active compound(s), filler, optional excipients and vehicle into a mixed state at a temperature above or within the melting range of the vehicle under agitation,
wherein said active compound(s) in said mixed state is dissolved or dispersed as solid particles within said vehicle,
and whereby agglomerates form,
followed by cooling of the agglomerates under agitation or by spreading of the agglomerates onto trays or the like whereby the vehicle solidifies.

One method of melt agglomeration is performed by melting a pharmaceutically acceptable vehicle, dissolution or dispersion of one or more active compounds and optional excipients in the melted vehicle and deposition of the thus prepared mixture on a particulate material, the filler, and subsequently the particles adhere to each other and form agglomerates. Alternatively, all ingredients are mixed at ambient temperature followed by heating to a temperature above the melting point of the vehicle which melts whereby agglomerates form. Combinations and variations of these methods are known to those skilled in the art. Hence, melt agglomeration is a convenient process for preparation of pharmaceutical formulations of active compounds as it is a robust and well-controllable process comprising few unit operations. A melt agglomerate is the product of a melt agglomeration process as described above.

U.S. Pat. No. 5,403,593 discloses a process for melt granulation comprising a hydrophilic cellulose ether polymer or a mixture thereof, a granulating medium having a melting range above 30° C. consisting of a lipid component and ethylene oxide polymers and mixtures thereof, and a therapeutically active medicament.

EP 0 841 062 A1 discloses a method for preparing a granular preparation by melt granulation of a powdered low-melting oily substance and a powdered medicine, the particles being coated with a finely powdered hydrophobic and oil-absorbing polymeric compound. As examples of a finely powdered hydrophobic and oil-absorbing polymeric compound is mentioned cellulose derivates such as ethyl cellulose. The obtained granules do not cake under heat and humid conditions. Further an unpleasant taste of the medicine can be masked.

EP 0 985 411 A1 discloses the preparation of solid oral dosage forms comprising sterol esters. A sterol ester adsorbate is formed by adding a surfactant to a melt of the sterol ester followed by addition of a support having a surface area of 100 to 350 square meters/gram in an amount sufficient to form a flowable powder. As examples of supports can be mentioned magnesium aluminosilicate, tricalcium phosphate and silicon dioxide. It is further disclosed that by use of tricalcium phosphate as support an adsorbate in form of an agglomerate was obtained, in contrast to the use of magnesium aluminosilicate or silicon dioxide as support, where adsorbates in form of free-flowing powders were obtained. The disclosed process is not a melt agglomeration as the surfactants used do not melt because they are liquid at ambient temperature.

U.S. patent application 2002/0160050 A1 discloses melt granulated compositions comprising one or more hydrophilic cellulose ether polymers, a hydrophilic melt binder and a therapeutically active ingredient. The disclosed granulated compositions are useful for the preparation of solid modified release dosage forms.

WO 01/41733 A2 discloses the preparation of granules where an active compound is dissolved in an oil and this mixture is subsequently mixed with silicon dioxide where-after the mixture is spread on a steel table, cooled and milled into granules.

In EP 448 091 A2 an active compound is dissolved in a fatty acid monoglyceride or polyoxyethylenesorbitan fatty acid ester and optionally the liquid solution is adsorbed onto a porous inorganic substance e.g. magnesium aluminate silicate. The disclosed process is not a melt agglomeration because the oil used as vehicle does not melt because it is liquid at ambient temperature.

GB 1442951 discloses melt granulates wherein silicon dioxide is used intragranularly as a loosening agent or tablet disintegrant as well as extragranularly as a flowing agent or glidant. The intragranular amount of silicon dioxide is less than 3% by weight of the granules.

FR 2 594 693 and FR 2 648 708 disclose processes for the manufacture of dry emulsions wherein an oil-in-water emulsion is solidified upon a mixture of a hydrophilic and a hydrophobic filler. This is not a melt agglomeration process as defined in this application as the active compound is dissolved in the aqueous part of the emulsion.

U.S. Pat. No. 5,403,593 discloses the use of silicon dioxide as extragranular glidant.

Melt granulation using an active compound, a meltable vehicle and a silicate is disclosed in several documents e.g. Gupta et al. in Pharm. Dev. Technol. 2001, 6, 563-572 and Gupta et al. in Pharm. Dev. Technol. 2002, 7, 103-112.

### DESCRIPTION OF THE INVENTION

The enhancement of oral bioavailability of poorly water soluble drugs as well as providing a fairly water soluble drug in a sustained release form remain some of the most challenging aspects of drug development and further development of the melt agglomeration technique may provide valuable tools for these aspects.

Thus the present invention relates to a new and useful agglomerated composition comprising:

a) one or more pharmaceutically acceptable vehicles having a melting temperature above ambient temperature;

b) one or more pharmaceutically active compounds;

c) a filler consisting of particular silicon dioxide.

It has surprisingly been found that the agglomerated compositions according to the invention can contain a high amount of vehicle and/or vehicle having active compounds dissolved or dispersed therein.

This surprising realization provides the advantage that pharmaceutical compositions for oral ingestion such as tablets or capsules prepared using the agglomerated compositions according to the invention having a higher content of vehicle and/or the active compound can be manufactured. Alternatively smaller tablets may be prepared with the following improved acceptance by the consumer and a reduced consumption of excipients, tablet additives, coatings etc. for the manufacturer. Further, a higher amount of vehicle may be incorporated into a pharmaceutical composition in order to improve the bioavailability of the active compound.

Further the agglomerated compositions according to the invention can easily be compressed into tablets. It is surprising that a melt agglomeration using silicon dioxide as filler provides an agglomerate since EP 985 411 discloses that by melting a sterol ester followed by coating said melt on a silicon dioxide support a free flowing powder was formed and not an agglomerate.

In another aspect the invention relates to a procedure for the preparation of the agglomerated composition.

In the present specification the term “melt agglomeration” is used for a process for preparing a material where a melt of a vehicle optionally comprising an active compound is deposited on a particulate filler material to enable the formation of an agglomerate. The process is also in the literature known under other terms e.g. “melt granulation”.

The term “vehicle” is intended to mean a compound or mixture of compounds functioning in melted state as solvent or dispersing medium for the active compound according to the invention. In melt agglomeration the vehicle also serves as a binder between different particles to enable the formation of the agglomerate.

The term “filler” is intended to mean a particulate inert material upon which the melted vehicle optionally comprising an active compound dissolved or dispersed therein is deposited.

The term “inert” is intended to mean that the material in question does not participate in any chemical reaction with other constituents of the mixture at the conditions applied during preparation and storage thereof.

The term “agglomerate” is used in the usual meaning i.e. a material composed of agglomerated primary particles. It is usually preferred to prepare agglomerates comprising active compounds before these are manufactured into pharmaceutical composition. Agglomerates provide several benefits compared to powders such as less dusting during handling thereof, excellent flowability and a locked mixing state wherein the various ingredients can not segregate.

As used herein, “particle size distribution” means the volume distribution of equivalent spherical diameters as determined by laser diffraction at 0.2 bar dispersive pressure in a Sympatec Helos equipment. “Median particle size”, correspondingly, means the median of said particle size distribution.

Silicon dioxide is a well-known compound for pharmaceutical use having a number of known uses. The pharmaceutical use of silicon dioxide has been described in the well recognized “Handbook of Pharmaceutical Excipients, 3^{rd} ed. 2000, Published by the American Pharmaceutical Association, 2215 Constitution Avenue, NW Washington, D.C. 20037-2985 USA and the Pharmaceutical Press, 1 Lamberth High Street, London, UK; as adsorbent, anticaking agent; emulsion stabilizer; glidant; suspending agent; tablet disintegrant; thermal stabilizer; viscosity-increasing agent. Despite of the wide use of silicon dioxide within the pharmaceutical area the use as filler in a melt agglomeration process is new.

It is surprising that a melt agglomerate having silicon dioxide as filler may be used for the manufacture of pharmaceutical compositions because one would expect that the active compound comprised in said agglomerate would not be released at a sufficiently high rate because the silicon dioxide does not dissolve in the gastrointestinal tract and it may even provide a viscous gel.

In melt agglomeration it is believed that a substantial part of liquid mixture comprising the melted vehicle and the active compound is deposited on the surface of the filler where it is permanently localized by the solidification that takes place during the cooling to ambient temperature, even though some vehicle and active compound may be adsorbed in the filler and localized inside the material.

This is in contrast to an adsorption process where a liquid is deposited on a material and essentially completely adsorbed into pores etc. in the material.

The silicon dioxide for use according to the invention can in principle be any particulate pharmaceutically acceptable silicon dioxide.

Usually it is preferred to use fillers having a relatively small particle size because small particles have a higher surface to mass ratio, and therefore small particles will usually be able to support higher amount of vehicle per mass unit. However, if the particle size is very low the melt agglomeration process may be difficult to control.

The particle size of the particulate silicon dioxide may according to the invention be selected among wide limits. According to the invention silicon dioxide materials may be used having median particle sizes in the range of 2-400 μm, preferably in the range of 5-250 μm more preferred in the range of 10-200 μm, even more preferred in the range of 10-100 μm, and most preferred in the range of 20-30 μm.

As examples of commercially available silicon dioxide products that may be used as filler according to the invention can be mentioned: Zeofree 5161A, Zeofree 5162, Zeofree 5175A and Zeopharm 80 all available from J. M. Huber (Hamina, Finland); Aeroperl 300, Sipernat 22, Sipernat 160PQ, Sipernat 700 and Sipernat 2200 available from Degussa (Frankfurt am Main, Germany); and Flo-Gard FFD available from PPG Industries (Pittsburgh, Pa., USA).

The vehicle for use according to the invention may in principle be any inert pharmaceutically acceptable compound being semisolid or solid at room temperature (25°) and which can be melted at a temperature above ambient temperature.

A suitable melting temperature for the vehicle is in the range of 37-200° C., preferred in the range of 40-150° C., more preferred in the range of 50-120° C. and most preferred in the range of 50-100° C.

As examples of vehicles according to the invention can be mentioned: polyethylene glycols, esters of polyethylene glycols, waxes, glycerides, fatty acid alcohols, fatty acids, sugar alcohols, vitamin E and derivatives of vitamin E.

The vehicle may even be a mixture of two or more vehicles.

The dissolution of the vehicle and/or the mixture of vehicle and active compound(s) in an aqueous medium may be fast or slow depending on the properties of the particular compounds and the particular aqueous medium. It will be appreciated that the terms “fast or slow” will relate to the intended use for said vehicle and/or mixture of vehicle and active compound(s). It is within the skills of the average practitioner to determine if a particular vehicle and/or mixture of vehicle and active compound(s) is (are) dissolved fast or slowly in a given aqueous medium using general knowledge and by performing routine experimentation.

The release of the active compound will be strongly influenced by the particular selected vehicle. Thus if a fast dissolving vehicle is selected the active compound will be released fast from the agglomerate when the agglomerate is dispersed in an aqueous environment, presumably because the vehicle will be fast dissolved thereby releasing the active compound. If a slow dissolving vehicle is selected, the active compound will be released slower from the agglomerate, presumably because the agglomerate will remain essentially intact and the active compound is released mainly by diffusion and dissolution from the surface of the granules. By selecting a vehicle having intermediate dissolution properties in water an agglomerate having intermediate release rate of the active compound may be obtained.

The release rate for a given combination of vehicle and active compound can easily be determined using routine experiments known as such.

The active compound can in principle be any compound having a biological activity that may be advantageous within the pharmaceutical area, and which compound can exert its activity in or can be absorbed from the gastrointestinal tract. Thus according to the invention active compounds may be compounds used in a treatment, prophylaxis or alleviation of a physical or mental condition or may even be a compound having a beneficial effect on the nutritional state of the recipient thereof, such as vitamins. The active compound may according to the invention even be a mixture of two or more active compounds.

As examples of active compounds can be mentioned organic molecules and salts such as: paracetamol, metoprolol, theophylline, acyclovir, atenolol, cimetidine, ranitidine, atovaquone, carbamazepine, danazol, glibenclamide, griseofulvin, ketoconazole, troglitazone, chlorothiazide, furosemide, cyclosporin A and itraconazole. Other examples of active compounds are inorganic molecules and salts such as: potassium salts such as potassium chloride; lithium salts such as lithium carbonate, lithium citrate and lithium sulphate; and iron salts such as ferrous sulphate, ferrous succinate, ferrous gluconate, ferrous fumarate and ferrous tartrate.

The active compound may be dissolved or dispersed in the melted vehicle thus forming a solid solution or solid dispersion with the vehicle upon cooling and solidification of the mixture.

Thus, for a given active compound a vehicle having the desired dissolving or dispersing properties in respect of the particular active compound should be selected. It is within the skills of the average practitioner to determine if a given vehicle has the desired propertied with respect to a given active compound.

The skilled person will appreciate that the particular intended active compound may pose certain limitations regarding the choice of vehicle that may be used for the particular agglomeration process. In particular a suitable vehicle for a given active compound may be selected taking due care to the melting point of the vehicle in order to select a vehicle that may be melted and agglomerated at a temperature where the active compound is not deteriorated to an unacceptable extend.

Depending on the intended use of the particular agglomerate the ratio of vehicle including dissolved or dispersed active compound to the filler can be varied between wide limits. Thus the amount of vehicle including dissolved or dispersed active compound may preferably constitute up to 75% by weight of the agglomerate. It is preferred that the relative amount of vehicle including dissolved or dispersed active compound is not too low in order to avoid large pharmaceutical compositions comprising said agglomerate. The preferred amount of vehicle including dissolved or dispersed active compound is in the range of 20-75% by weight of the agglomerate, preferably in the range of 40-70% by weight of the agglomerate, more preferred in the range of 50-70% by weight of the agglomerate. In a particular embodiment the intragranular amount of the silicon dioxide filler is at least 5% by weight of the agglomerate, more particularly at least 10%, even most particularly at least 15% and most particularly at least 20%.

The ratio of active compound(s) to vehicle is determined by the nature and properties of the given vehicle and active compound(s). In one embodiment the ratio is high in order to be able to prepare an agglomerate having a high amount of active compound per mass unit of the agglomerate. In another embodiment the ratio is low in order to improve the release of an active compound and thereby increasing the bioavailability of said active compound. In another embodiment the ratio is low and the vehicle is insoluble or has a low solubility in water in order to provide a sustained release of an active compound over a prolonged period of time and thereby providing a controlled release formulation of said active compound.

Pharmaceutically accepted additives or excipients may also be added to the mixture of vehicle and active compound(s), such as surfactant, solubility enhancer, stabilizer, preservative, fillers other than silicon dioxide etc., in order to influence the properties of the agglomerate, or in order to facilitate the manufacturing, as it will be known from recognized handbooks and textbooks within the area.

A preferred example of an optional filler to be added to the mixture of vehicle and active compounds in addition to silicon dioxide is lactose.

In principle agglomerates according to the invention may be prepared using procedures known within the area for melt agglomeration. Exemplary of apparatus, which may be used are low shear mixers, high shear mixers, fluid beds, fluid bed granulators, rotary fluidised beds and drum granulators.

In one embodiment the agglomerate is prepared by melting the vehicle, dissolving or dispersing the active compound in the melt, and spraying or pouring the melt on the particulate silicon dioxide. Alternatively, the filler and active compound are mixed whereafter the melted vehicle is sprayed or poured onto the mixture. The spraying or pouring step may be performed in accordance with known procedures.

In another embodiment all constituents of the agglomerate are added to a high shear mixer, optionally provided with a heating jacket. By operating the high shear mixer the friction heat and heat supplied by the heating jacket will melt the vehicle, which subsequently dissolve or disperse the active compound and deposits at the silicon dioxide. This method is a very attractive method for melt agglomeration, because the method is fast and easy to perform.

In the melt agglomeration processes the prepared agglomerate may be influenced by several process variables such as temperature of vehicle, filler and heating jacket; the impeller speed, time of treatment etc. The skilled person can using simple routine experiments determine suitable parameters for an intended melt agglomeration process using a given active compound, filler and vehicle, with use of a particular given suitable equipment.

In a particular embodiment of the invention the agglomerates formed have median particle sizes of at least 50 μm, more particularly in the range of 50-1000 μm, even more particularly in the range of 70-700 μm, yet even more particularly in the range of 80-500 μm, and most particularly in the range of 90-300 μm.

Agglomerates according to the invention may be used for the preparation of pharmaceutical compositions for oral administration according to well known procedures. Pharmaceutical compositions may be prepared by mixing agglomerate with usual pharmaceutically acceptable excipients, followed by preparing the composition using said mixture.

Preferred pharmaceutical composition for oral administration according to the invention are tablets and capsules.

Tablets may be prepared using procedures known as such, such as mixing the agglomerate according to the invention with known excipients usually used for tablets, and pressing the resulting mixture into tablets. The tablets may or may not be coated according to well-known procedures.

Capsules may be prepared using procedures know as such, for example mixing an agglomerate according to the invention with suitable excipients, and filling the mixture into suitable capsules, such as gelatine capsules.

In one preferred embodiment a pharmaceutical composition is prepared using an agglomerate according to the invention comprising an active compound and a water soluble vehicle. The pharmaceutical composition will provide the active compound for fast and high bioavailability of the active compound after ingestion of the pharmaceutical composition.

In another preferred embodiment a pharmaceutical composition is prepared using an agglomerate according to the invention comprising an active compound and a vehicle which is insoluble or has a low solubility in water. The pharmaceutical composition will provide a sustained release of the active compound over a prolonged period of time.

It may even be possible to prepare a pharmaceutical composition comprising two or more different agglomerates. These two or more agglomerates may comprise same active compound but different vehicles, thus providing differing release rates of the active compound from the two or more agglomerates, in order to provide a pharmaceutical composition having a particular desired release profile of the active compound. Alternatively the two or more agglomerates may comprise different active compounds. The skilled person will appreciate that other combinations may be used for providing a particular desired effect.

The invention will now be illustrated further by examples, which should not be regarded as limiting for the invention.

### EXAMPLES

In the following examples agglomerates and pharmaceutical formulations were prepared as formulations containing active compounds as well as placebo formulation i.e. without an active compound. However, it will be evident that the disclosed placebo examples which demonstrate the manufacture of melt agglomerates of the invention could likewise be performed using a mixture of a vehicle and one or more active compounds instead of a vehicle without active compound.

### Example 1

Placebo agglomerate consisting of 67% vehicle and 33% silicon dioxide.

A semi-solid solubility enhancing vehicle consisting of Macrogol 1500 was melted, the temperature of the melt was adjusted to 60° C. and added to VP Aeroperl®300 Pharma (silicon dioxide) during agitation in a high shear mixer.

The product was allowed to cool to room temperature, and appeared as a homogeneous agglomerate.

### Example 2

Placebo agglomerate consisting of 61% vehicle and 39% silicon dioxide.

A semi-solid solubility enhancing vehicle consisting of Macrogol 1500 was melted, the temperature of the melt was adjusted to 60° C. and added to Sipernat ®700 (silicon dioxide) during agitation in a high shear mixer.

The product was allowed to cool to room temperature, and appeared as a homogeneous agglomerate.

### Example 3

Placebo agglomerate consisting of 65% vehicle and 35% silicon dioxide.

A semi-solid solubility enhancing vehicle consisting of Macrogol 1500 was melted, the temperature of the melt was adjusted to 60° C. and added to Flo-gard FF-D® (silicon dioxide) during agitation in a high shear mixer.

The product was allowed to cool to room temperature, and appeared as a homogeneous agglomerate.

### Example 4

Placebo Tablets

An agglomerate consisting of 66% vehicle and 34% silicon dioxide was prepared as follows.

A semi-solid solubility enhancing vehicle consisting of 70% (w/w)Macrogol 1500 and 30% poloxamer 188 was melted, the temperature of the melt was adjusted to 60° C. and added to Sipernat 160PQ® (silicon dioxide) during agitation in a high shear mixer.

The agglomerate was allowed to cool to room temperature followed by addition of filler (Avicel PH 102), disintegrant (Ac-Di-Sol) and antisticking agent (magnesium stearate). The mixture was compressed to tablets with a weight of approximately 300 mg.

### Example 5

Formulation example. Placebo tablets.

A semi-solid solubility enhancing vehicle consisting of cetylanum emulsifying wax was melted and, subsequently, added to Sipernat 160PQ (silicon dioxide) during agitation in a high shear mixer. The formulation consisted of 64% vehicle and 36% Sipernat 160PQ (silicon dioxide). The agglomerate was allowed to cool to ambient temperature followed by addition of filler (Avicel PH200 and lactose 350 Mesh), disintegrant (Ac-Di-Sol) and antisticking agent (Magnesium stearate). The mixture was compressed to tablets with a weight of approximately 377 mg.

### Example 6

Instant Release Formulation

A semi-solid solubility enhancing vehicle consisting of PEG 1500 was melted at 70° C. and triamterene (a poorly soluble drug) was dispersed in the liquid vehicle. The dispersion was added to VP Aeroperl 300 (silicon dioxide) at 160 rpm in a high shear mixer. The blend was granulated at 800 rpm until a suitable particle size was obtained (the median of the volume size distribution was 56 μm). A placebo granulate containing PEG 1500 and Aeroperl was prepared by the same procedure. Finally, a standard solid dispersion containing triamterene and PEG 1500 was produced by dispersing triamterene in the melted vehicle and allowing the dispersion to cool in a thin layer. The material was subsequently milled to a suitable particle size.

All three formulations were filled into small capsules and administered orally to groups of five rats. The exact compositions of the formulations are shown in Table 1. The rats were kept separately in metabolism cages with 30 ml of drinking water available and the amount of urine excreted during 16 hours was determined (triamterene is a diuretic acting drug). The amount of urine excreted is shown in Table 2. The data illustrates that including silicon dioxide in the formulation does not impair the absorption of drug from the solid dispersion.

TABLE 1
Compositions of formulations for rat study.

| | | | Silicon | Total |
|---|---|---|---|---|
| | Triamterene | PEG 1500 | dioxide | amount |
| Formulation | (mg) | (mg) | (mg) | (mg) |
| New formulation | 2.5 | 6.5 | 3.5 | 12.5 |
| Standard solid | 2.5 | 6.5 | — | 9 |
| dispersion | | | | |
| Placebo | — | 6.5 | 3.5 | 10 |

TABLE 2
Amount of urine excreted in rat study.

| | Urine excreted |
|---|---|
| Formulation | (ml) |
| New formulation | 20.8 |
| Standard solid dispersion | 22.4 |
| Placebo | 9.6 |

### Example 7

Controlled Release Pharmaceutical Formulation

A semi-solid release rate controlling vehicle consisting of stearic acid was melted at 60° C. and potassium chloride (the model drug) was dispersed in the liquid vehicle. The dispersion was added to VP Aeroperl 300 (silicon dioxide) at 160 rpm in a high shear mixer. The blend was granulated at 800 rpm and a particle size of 293 μm was obtained. The formulation contained 10% potassium chloride, 55% stearic acid and 35% silicon dioxide.

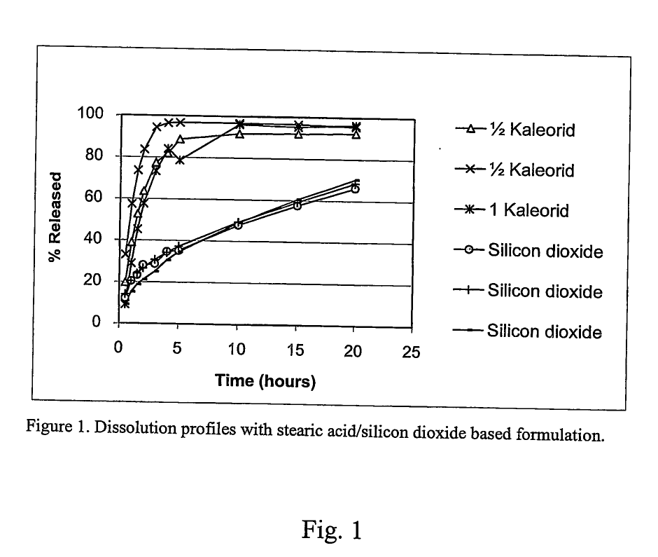

In vitro dissolution tests were performed to document the prolonged release. Amounts of 500 mg formulation containing 50 mg potassium chloride were filled into hard gelatine capsules. The formulations were tested with a paddle dissolution equipment in 900 ml water at 37° C. and a paddle rotation speed of 50 rpm. Detection was performed with a conductivity measuring probe. A Kaleorid tablet (750 mg KCl) and two halves were also analysed for comparison. Typical release profiles are shown in Figure 1.

It can be seen from the figure that the silicon dioxide/stearic acid based formulation give rise to a different kind of profile compared to the Kaleorid formulation which shows a much faster release. In Kaleorid the potassium chloride is distributed in an insoluble matrix and the formulation is meant to be swallowed unbroken. The drug release is controlled by diffusion in the core material. It can be concluded that the silicon dioxide formulation possesses good controlled release properties.

1. A melt agglomerated composition comprising:
a) one or more pharmaceutical acceptable vehicles having a melting temperature above ambient temperature;
b) one or more active compounds dissolved or dispersed in the one or more pharmaceutical acceptable vehicles; and
c) a filler consisting of particulate silicon dioxide.

2. The composition according to claim 1, wherein the intragranular amount of silicon dioxide is at least 5% by weight of the granules.

3. The composition according to claim 1 or 2, wherein the vehicle has a melting temperature in the range of 37-200° C., preferably in the range of 50-100° C.

4. The composition according to any of claims 1 to 3, wherein the amount of vehicle and active compound accounts for 20-75% b.w. of the composition.

5. The composition according to claim 4, wherein the amount of vehicle and active compound accounts for 40-70% b.w. of the composition.

6. The composition according to claim 5, wherein the amount of vehicle and active compound accounts for 50-70% b.w. of the composition.

7. The composition according to any of claims 1-6, wherein the particulate silicon dioxide has a median particle size in the range of 2-400 μm.

8. The composition according to claim 7, wherein the particulate silicon dioxide has a median particle size in the range of 10-100 μm.

9. The composition according to claim 8, wherein the particulate silicon dioxide has a median particle size in the range of 20-30 μm.

10. The composition according to any of the claims 1-9, wherein the vehicle is selected among: polyethylene glycols, esters of polyethylene glycols, waxes, glycerides, fatty acid alcohols, fatty acids, sugar alcohols, vitamin E, derivatives of vitamin E, and mixtures thereof.

11. The composition according to any of the claims 1-10, wherein the agglomerates formed have median particle sizes of at least 50 μm.

12. The composition according to any of the claims 1-11, wherein the active ingredient is potassium chloride.

13. Use of a composition according to any of the claims 1-12, for the manufacture of a pharmaceutical medicament for oral administration.

14. Use according to claim 13, wherein the pharmaceutical medicament is a tablet or a capsule.

15. Use according to claim 13 or 14, for the manufacture of a pharmaceutical composition providing controlled release of the active compound.

16. Use according to claim 13 or 14, for the manufacture of a pharmaceutical composition providing instant release of the active compound.

17. A process for the preparation of an agglomerated material composition according to any of claims 1-12, comprising the following steps:
i) heating a composition comprising:
a) one or more pharmaceutical acceptable vehicles being solid at room temperature and having a melting temperature above 37° C.; and
b) one or more active compounds being soluble in the melted vehicle;
ii) adding said composition to a filler consisting of particulate silicon dioxide while mixing the mixture to provide the agglomerate as a substantially homogeneous composition; and
iii) cooling the composition to room temperature.

18. A process for the preparation of an agglomerated material composition according to any of claims 1-12, comprising the following steps:
i) providing a mixture comprising:
a) one or more pharmaceutical acceptable vehicles being solid at room temperature and having a melting temperature above 37° C.;
b) one or more active compounds being soluble in the melted vehicle; and
c) a filler consisting of particulate silicon dioxide;
ii) mixing and heating the mixture until formation of the agglomerate as a substantially homogeneous composition; and
iii) cooling the composition to room temperature.

19. A process according to claims 17 or 18, wherein the agglomeration is performed in a device selected from the group comprising low shear mixers, high shear mixers, fluid beds, fluid bed granulators, rotary fluidised beds and drum granulators.

20. Pharmaceutical composition comprising an agglomerate according to any of the claims 1-12.

21. Pharmaceutical composition according to claim 20, wherein the pharmaceutical composition is in the form of a tablet or a capsule.

22. Pharmaceutical composition according to claim 20 or 21, wherein the pharmaceutical composition provides controlled release of the active compound.

23. Pharmaceutical composition according to claim 20 or 21, wherein the pharmaceutical composition provides instant release of the active compound.
